(19) United States
(12) Patent Application Publication
(10) Pub. No.: US 2021/0221777 A1
(43) Pub. Date: Jul. 22, 2021

(54) NOVEL COMPOUNDS RELATED TO MYCOTHIAZOLE AND METHODS OF TREATING CANCER

(71) Applicants: Tyler A. Johnson, San Rafael, CA (US); Colon V. Cook, San Rafael, CA (US); Nicole L. McIntosh, Novato, CA (US); Joseph D. Morris, San Rafael, CA (US); Frederick A. Valeriote, Grosse Pointe Shores, MI (US); JiaJiu Shaw, Frisco, TX (US); Phillip Crews, Santa Cruz, CA (US); Mani Maheshwari, Farmington Hills, MI (US)

(72) Inventors: Tyler A. Johnson, San Rafael, CA (US); Colon V. Cook, San Rafael, CA (US); Nicole L. McIntosh, Novato, CA (US); Joseph D. Morris, San Rafael, CA (US); Frederick A. Valeriote, Grosse Pointe Shores, MI (US); JiaJiu Shaw, Frisco, TX (US); Phillip Crews, Santa Cruz, CA (US); Mani Maheshwari, Farmington Hills, MI (US)

(21) Appl. No.: 17/149,203

(22) Filed: Jan. 14, 2021

Related U.S. Application Data
(60) Provisional application No. 62/962,175, filed on Jan. 16, 2020.

(51) Int. Cl. C07D 277/30; A61P 35/00

(52) U.S. Cl. CPC C07D 277/30; A61P 35/00

(57) ABSTRACT

A pharmaceutically active mycathiozole analog or salt thereof is derived from naturally occurring mycathiozole. The analog or salt is useful in cancer treatment.

### CROSS-REFERENCE TO RELATED APPLICATIONS

This application is a completion application of co-pending U.S. Provisional patent Application Ser. No. 62/962,175, filed Jan. 16, 2020, NOVEL COMPOUNDS RELATED TO MYCOTHIAZOLE AND METHODS OF TREATING CANCER, the entire disclosure of which is hereby incorporated by reference in its entirety.

### BACKGROUND OF THE INVENTION

### 1. Field of the Invention

The present invention is related to a novel marine small molecule, mycothiazole, and related derivatives as well as methods of treating cancer.

### 2. Prior Art

In conclusion, the present invention discloses several novel compounds, pharmaceutical dosage forms, and methods of treating cancer. The present invention also discloses a method of isolation and storage method for extended shelf lives of the compounds. Although the description above contains many specificities, these should not be construed as limiting the scope of the invention but as merely providing the illustrations of some of the presently preferred embodiments of this invention. Thus, the scope of this invention should be determined by the appended claims and their legal equivalents, rather than by the specificities or examples given.

The sponge-derived mycothiazole (1) chemotype¹ has drawn considerable attention over the last 30 years based on the precise chemical assignment of its molecular structure,²⁻³ and discoveries of its unique mechanism of action,⁴⁻⁵ which are responsible for its impressive cytotoxicity (IC₅₀ 0.36-13.8 nM) against a wide variety of solid tumor cell lines.⁵⁻⁸ To date, three total synthesis^{3,9-13} and four partial synthesis¹⁴⁻¹⁷ have been reported for 1 by five different research groups.³ Other noteworthy structural developments of 1 include: a) assignment of the 8R absolute configuration at the C(8)-OH stereocenter which was proposed by comparing optical rotation values for natural 1 ([α]_{D}=−3.0)¹ with synthetic samples of 1 (8R ([α]_{D}=−26.0 and 8S ([α]_{D}=+22.0)¹⁰ and b) revision of the E stereochemistry originally reported for the natural 1¹ at positions C-14/C-15 to Z.^{2,6} It is also well documented that compound 1 is labile.^{3,4,6,9,10}

Despite the need for next generation analogs of 1 with increased stability, and or cytotoxicity involving structure activity relationship (SAR) data, only six distinct structural analogs of 1 have been reported and their structures are shown in FIG. 1.^{4,6,18-19} Several trends exist regarding the cytotoxic SAR data for 2-7 against tumor cell lines. For example, Manta et al.,¹⁸ debuted the first-generation synthetic analogs (3-6) devoid of the unstable diene system at C-4, C-19. Modifications to the thiazole ring introducing a thiazoline (4) or oxazoline (5) either increased or decreased potency against human colon tumor (HCT-15) cell lines by three fold (4, GI₅₀=13 μM; 5, GI₅₀=150 μM), while an oxazoline with the elimination of the C16-C18 side chain reduced potency by nearly ten fold (6, GI₅₀=430 μM), in comparison to 1 (IG₅₀=44 μM).¹⁸ The first reported semi-synthetic analog of 1, 8-O-acetyl-mycothiazole (2), demonstrated potency (IC₅₀˜1 nM) against hypoxia-induced factor (HIF-1) signaling in T47D breast tumor cells, on par with 1 (IC₅₀˜1 nM).⁴ Mycothiazole-4,19-diol (7), the first analog reported alongside the isolation of natural 1, was at least 1000 times less potent then 1 or 2 at suppression of hypoxia-induced HIF-1 activation in T47D breast tumor cells.⁴

### SUMMARY OF THE INVENTION

A compound of formula (I), formula (II), or pharmaceutically acceptable salt thereof is disclosed along with their use in cancer treatment,

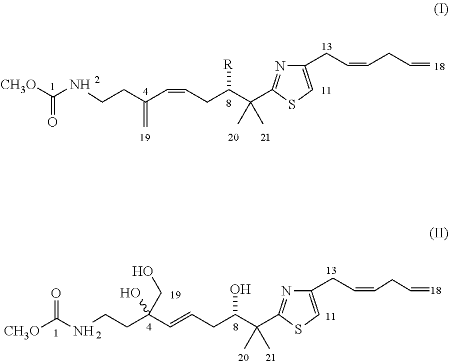

wherein R is OH, O-acetate, O—CH₃, F, Cl, Br, I, or an antibody.

### BRIEF DESCRIPTION OF THE DRAWING

FIG. 1 depicts the natural occurring mycothiazole denoted as (1) and its analogs; and

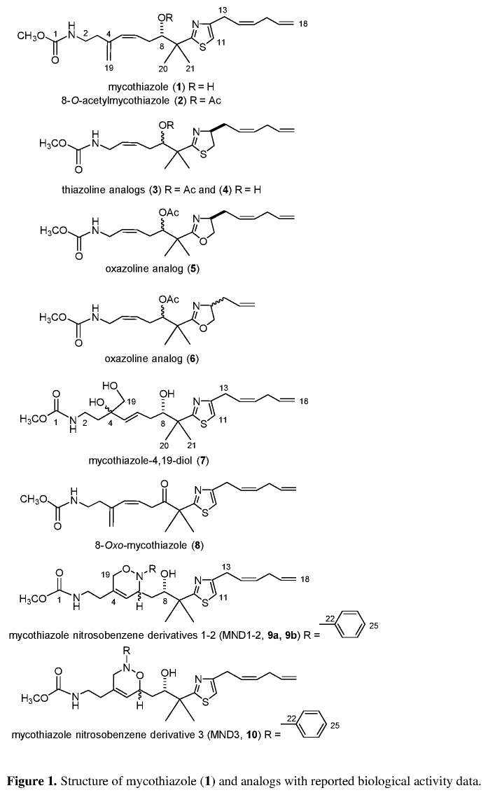

FIG. 2 is a summary of LCMS data to follow degradation of mycothiazole (I) to mycothiazole-4,19-diol (7).

### DESCRIPTION OF THE INVENTION

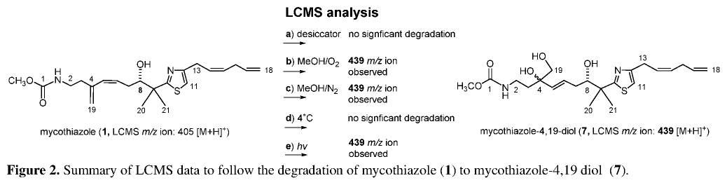

Recent work in our laboratory using HPLC during the scale up isolation of mycothiazole (1) hinted that mycothiazole-4,19-diol (7)⁶ may also be an artifact of isolation encountered during routine purification of degraded 1. To test this hypothesis, we subjected freshly purified 1 to a variety of sample storage experimental conditions (a-e) summarized in Table 2. Analytical (LCMS) data was used to determine purity outcomes of 1 in terms of its observed degradation which is summarized at the bottom of Table 2 and in FIG. 2. The LC (UV-PDA) data indicated that compound 1, generated 500000 AU for samples a-d. However, sample e generated 280000 AU for 1, suggesting severe degradation of 1. The MS-TIC data showed a similar trend which included observation of: a) approximately 20% relative abundance of m/z ion impurity peaks for our control experiment indicating minimal degradation, b) and c) 45% relative abundance of m/z ion impurity peaks, indicating partial degradation, d) 20% relative abundance of m/z ion impurity peaks indicating minimal degradation and e) 60% relative abundance of m/z ion impurity peaks indicating severe degradation. Furthermore, the diagnostic MS m/z ion peak (439, [M+H]⁺)⁶ characteristic of 7 was also observed in samples b-c and e, but not in a or d, as shown in FIG. 2. There were several other impurity (m/z ion) peaks detected in our LCMS analysis of samples b, c and e shown in Figures S8, S9 and S11. Selected examples included molecular ion peaks of 421 m/z and 437 m/z. We were unable to pursue full or even partial structure elucidation of these components or the observed 439 m/z ion peak, using NMR studies, due to insufficient material. Therefore, we postulate that the 439 m/z ion peak observed in our experiments here, suggests that mycothiazole 4-19 diol (7) is an artifact of isolation encountered during the routine purification of degraded samples of 1.

TABLE 1
Cytotoxicity of 1 and Analogs Vs. Tumor Cell lines.

| | PANC-1 | HepG2 | HCT 116 |
|---|---|---|---|
| compound | IC₅₀ (nM) | IC₅₀ (nM) | IC₅₀ (nM) |
| mycothiazole (1) | 0.160 ± 15^{a} | 0.270 ± 6.1^{a} | 0.350 ± 12^{a} |
| | | | 3.8 ± 0.24^{b, c} |
| 8-O-acetyl-mycothiazole (2) | 1.30 ± 0.040^{a} | NT | NT |
| mycothiazole-4,19-diol (7) | NT | NT | >1000.0^{a} |
| 8-Oxo-mycothiazole (8) | 2000.0 ± 0.15^{a} | NT | NT |
| mycothiazole MND1 (9a) | >1000.0^{a} | NT | NT |
| mycothiazole MND2 (9b) | >1000.0^{a} | NT | NT |
| mycothiazole MND3 (10) | >1000.0^{a} | NT | NT |

^{a}This work. ± SEM data includes N = 2.
^{b}Sashidhara et al., 2009, J. Nat. Prod. 72, 588.
^{c}Data may be inaccurate due to compound degradation. Not tested (NT).

TABLE 2
Sample Storage Experimental Conditions, Analytical data
and Purity Outcomes Leading to Transformation of 1 to 7.

| sample storage | a) standard protocol | b) MeOH—O₂ | c) MeOH—N₂ | d) refrigeration | e) direct light |
|---|---|---|---|---|---|
| experimental conditions | (desiccator) | (Air stream) | (N₂ stream) | (4° C.) | (hv) |
| vial type | amber | amber | amber | amber | clear |
| evap. solvent | ≈95% CH₃CN^{a} | MeOH | MeOH | ≈95% CH₃CN^{a} | ≈95% CH₃CN^{a} |
| evap. method | N₂ stream | Air stream | N₂ stream | N₂ stream | N₂ stream |
| storage gas | Argon | none | none | Argon | none |
| storage conditions | dark desiccator - under | room temp. | room temp. | 4° C. | direct light (hv) |
| | vacuum (24 hrs) | (22° C., 24 hrs) | (22° C., 24 hrs) | (24 hrs) | (24° C., 24 hrs) |
| analytical LCMS data | 500000 AU | 500000 AU | 500000 AU | 500000 AU | 280000 AU |
| UV-PDA^{b,} * | | | | | |
| MS − TIC^{c,} * of visible | ~20% relative | ~45% relative | ~45% relative | ~20% relative | ~60% relative |
| impurity peaks | abundance | abundance | abundance | abundance | abundance |
| | of m/z ions | of m/z ions | of m/z ions | of m/z ions | of m/z ions |
| purity outcomes of 1 | minimal | partial | partial | minimal | severe |
| involving its degradation | | | | | |

^{a}Contains ~5% water.
^{b}Maximum absorbance units (AU) for the reference compound mycothiazole (1) observed from ultraviolet-photo diode array (UV-PDA) detection.
^{c}Relative absorbance of average (~) mass spectrometry − total ion current (MS − TIC) impurity m/z ion peaks detected versus reference compound 1.
*For more details regarding LCMS data of degradation experiments a-e, see FIGS. S7-S11 in the supporting information.

Overall the results in Table 2 and FIG. 2 suggested: 1) exposure of 1 to protic solvents (e.g. methanol) and direct light may accelerate its degradation, 2) using amber vials with aprotic solvents to collect and transfer 1 (e.g. CH₃CN, or CH₂Cl₂) along with storage in a dark desiccator under vacuum or refrigeration may impede it's degradation and 3) compound 7 may not be a bonafide natural product, rather an artifact that exists alongside other oxidized products associated with the degradation of 1. At this juncture, it became clear samples of 1 previously evaluated for their cytotoxicity against tumor cell lines⁵⁻⁸ may have been partially or severely degraded during the time of their bioassay due to the limited shelf life we observed for 1, unless properly stabilized.⁴ This factor could explain the discrepancy in potency and or selectivity of 1 against tumor cell lines we observed for our results shown in Table 1 and Table S1 compared to data from 2009.⁷

Next, we turned our attention to re-evaluating 1 based on its past optical rotation (OR) data shown in Table 3. The first total synthesis of 1 was done by Shiori.^{9,10} The 8R absolute configuration at the C(8)-OH stereocenter of 1 was proposed based on comparison of OR values reported for natural 1 (entry 1, [α]_{D}²³=−3.0, CHCl₃)¹ which matched most closely with the synthetic samples of 1 for 8R (entries 2-3, [α]_{D}²³=−26.0, CHCl₃) versus the 8S enantiomer (entry 4, [α]_{D}²³=+22.0, CHCl₃).¹⁰ However, this version of the 8R enantiomer of 1 reported the E stereochemistry at positions C-14/C-15 based on the original report of natural 1 isolated from C. mycofijiensis found in Vanuatu.¹ A structural reassignment of natural 1, was made in 2006⁶ revising the E stereochemistry at positions C-14/15 to Z, yet it reported similar OR data for natural 1 shown in entry 5, ([α]_{D}²³=−3.5, CHCl₃). Interestingly, a Tongan sample of C. mycofijiensis was reportedly the source of the 8R enantiomer of 1 with a larger negative OR value shown in entry 6 ([α]_{D}²⁰=−19.3, CHCl₃).⁵ Why these data sets varied widely for allegedly the same compound 1 is not entirely clear. Previous reports suggested the discrepancy observed in the original OR data set obtained for natural 1 listed in entry 1 versus the two synthetic enantiomers of 1 listed in entries 2-4 was due to the possibility of contamination with artifacts that existed in samples of natural 1 when originally measured.⁹⁻¹² Upon reevaluation of freshly purified 1, confirmed by ¹H NMR (C₆D₆) 24 hrs prior to and after obtaining it's OR data (see Figure S12), we anticipated measuring a negative value closer to entries 2 and 7 reported by others for the correct total synthesis of the 8R enantiomer.^{3,9-12} To our surprise, the value we measured (entry 8) was [α]_{D}²⁵=+25.78 (CHCl₃), which was intriguing. This suggested a reassignment of the 8R configuration assigned at the C(8)-OH stereocenter of 1 to the 8S for all of our previous samples of 1 that were derived from Vanuatu specimens of C. mycofijiensis.^{1,4,6} To investigate this further we prepared a sample of semi-oxidized 1 (entry 9), by immersing pure 1 in 2000 μl methanol, 400 μl water, then evaporating it slowly over an air stream for 48 hrs. ¹H NMR indicated partial degradation of 1 as shown in Figure S13 before measuring the data set in entry 9 ([α]_{D}²⁵=+12.40, CHCl₃). This suggested that as 1 is oxidized, its optical rotation value becomes more negative. Such results are consistent with our theory that as 1 degrades; compound 7 may emerge into solution in greater quantities alongside 1. Since 7 has an additional undefined chiral center (C-4), the existence of 7 and or other degradation artifacts like it, may be responsible, at least partially, for what we believe to be inaccurate OR values previously reported for pure natural 1 derived from Vanuatu samples of C. mycofijiensis (entries 1, 5).^{1,2,4,6}

TABLE 3
Comparison of Reported Optical Rotation Values of Natural Mycothiazole (1) Versus Synthetic Samples of 1.

| entry | compound | C-8 | C14-C15 | solvent | reference and notes |
|---|---|---|---|---|---|
| 1 | natural 1 | R | E | [α]²³_{D} = −3.8 (c 2.9, CHCl₃) | Crews et al., 1988, J. Am Chem. Soc.^{a} |
| 2 | synthetic 1 | R | E | [α]²³_{D} = −26.0 (c 0.6, CHCl₃) | Shiori et al., 2000, Org. Lett.^{a, b, c} |
| 3 | synthetic 1 | R | E | [α]²³_{D} = −26.0 (c 0.64, CHCl₃) | Shiori et al., 2003, Tetrahedron^{a, b, c} |
| 4 | synthetic 1 | S | E | [α]²³_{D} = +22.5 (c 0.53, CHCl₃) | Shiori et al., 2003, Tetrahedron^{a, b, c} |
| 5 | natural 1 | R | Z | [α]²³_{D} = −3.5 (c 0.6, CHCl₃) | Crews et al., 2006, J. Nat. Prod.^{a} |
| 6 | natural 1 | R | Z | [α]²⁰_{D} = −19.3 (c 0.93, CHCl₃) | Miller et al., 2012, Marine Drugs^{a} |
| 7 | synthetic 1 | R | Z | [α]²³_{D} = −21.4 (c 0.47, CHCl₃) | Hale et al., 2015, Org. lett. |
| 8 | natural 1 | S | Z | [α]²⁵_{D} = +25.78 (c 1.11, CHCl₃) | This work.^{d} |
| 9 | semi-oxidized 1 | S | Z | [α]²⁵_{D} = +12.40 (c 1.57, CHCl₃) | This work.^{f} |

^{a}Purity of natural mycothiazole (1) not reported by ¹H NMR (C₆D₆) 24 hrs prior to conducting optical rotatory dispersion (ORD) experiment(s).
^{b}Synthetic samples of 1 reported to be labile.
^{c}Natural sample of 1 provided for comparative ORD measurements with synthetic (1, 8R, and 8S) were unsuccessful due to degradation of natural 1.
^{d}Purity of 1 confirmed by ¹H NMR (C₆D₆) 24 hr prior to obtaining ORD data set(s), see FIG. S12 in the supporting information.
^{f}Impurity peaks indicating semi-oxidized (degraded) 1 are confirmed by ¹H NMR (C₆D₆) 24 hrs prior to running ORD experiment(s), see FIG. S13 in the supporting information.

Our final approach reinvestigating 1 involved exploring the relevance of the C(8)-OH stereocenter and labile C-14/C-19 diene system for their SAR significance related to the cytotoxicity of 1 against tumor cell lines. We began with freshly purified 1 to generate 8-O-acetyl-mycothiazole (2) by semi-synthesis as reported previously.⁴ Preparation to provide pure 2 proceeded in a straightforward fashion as outlined in the experimental section and or scheme 51 of the supporting information. The new sample of 2 was preserved according to our standard storage protocol for 1 (Table 2a). ¹H and ¹³C NMR data (shown in Figure S14-S15) was consistent with previous reports for 2.⁴ Bioassay evaluation of 2 shown in Table 1 against pancreatic (PANC-1) tumor cell lines (IC₅₀=1.3 nM, PANC-1), was ten fold less potent than 1 (IC₅₀=0.160 nM). Nevertheless, compound 2 may be a more attractive therapeutic lead structure as preservation studies using ¹H NMR in Figure S16 revealed compound 2 was more stable than 1, for ≥6 months. This may be due to the C(8)-OAc serving as a protecting group. Fresh 1 was used to generate a novel achiral analog, 8-oxo-mycothiazole (8) through semi-synthesis using the Dess-Martin periodinane reagent.²¹⁻²² Preparation to provide 8 was relatively straight forward as outlined in the experimental section and in scheme S1. The structural assignment of 8 and other new analogs were confirmed using HAESIMS, 1D and 2D NMR data shown in Tables S3-S7 and Figures S17-S31. Surprisingly, the cytotoxicity of 8 against pancreatic (PANC-1) tumor cells lines (IC₅₀=2000.0 nM) was 10,000 times less potent then 1. This underscores the importance of the C(8)-OH stereocenter in terms of its ability to significantly effect the cytotoxicity of this unique structural class against tumor cell lines. Lastly, we investigated analogs with improved stability devoid of the labile C-4/C-19 diene system by generating mycothiazole nitrosobenzene derivatives (MND) 1-2, (9a-9b) and MND3 (10). Preparation and purification are discussed in the experimental and resulted diastereomers 9a and 9b and pure 10 only. Compounds 9-10 were more stable then 1, unfortunately they were 10,000 times less potent (IC₅₀=>1000.0 nM, PANC-1) and on par with 8.

A collection of noteworthy conclusions can be drawn from our reinvestigation of 1. Significantly, evaluation of fresh 1 has shown a tenfold increase in its potency (IC₅₀=160-360 pM) against pancreatic (PANC-1), liver (HepG2), and colon (HCT-116) tumor cell lines. These data heighten the impact of findings from this project because few natural products have been described with picomolar potency against tumor cell lines. Selected such examples include the cryptophycins,²³ lomaiviticin,²⁴ and psymberin.²⁵ Further, some of the members of this list have drawn considerable attention from the medicinal chemistry community for their development as antibody drug conjugate (ADC) therapeutic leads.²⁶⁻²⁷ Each of these compounds plus mycothiazole possess structural novelty alongside spectacularly potent cytotoxicity.

It is important to underscore that mycothiazole (1) is unstable even when stored at 4° C. Alternatively, pure 1 can have a shelf life of 3-6 weeks by using the two storage protocols (a or d) outlined in Table 2. The labile nature of 1 may have influenced the accuracy of its cytotoxicity profiles reported in the past against a large panel of tumor cell lines.⁵⁻⁸ Another outcome of the instability of 1 is that it is converted to mycothiazole-4,19-diol (7) either during the isolation or storage as shown in FIG. 2.

Some additional, final observations are as follows. Based on the results obtained here, we now propose that the previously assigned 8R absolute configuration of 1 should be revised to 8S, but only for our samples of 1 isolated from Vanuatu specimens of C. mycofijiensis.^{1,2,4,6} The SAR studies reported here have provided fresh insights through evaluation of a mini library consisting of 1 and six analogs, 2b, 7, 8, 9a, 9b, 10 against three tumor cell lines (Table 1). It is clear that the penta-2,4-dien-1-ol residue, a unique structural component of 1, is required to impart its picomolar cytotoxicity. To date there are no reports of the total synthesis of 1 with the revised 8S and C14/C15 double bond Z configuration and the results shown here indicate the pressing need for this action. Going forward it will be interesting to evaluate both enantiomers (8S, 8R) of synthetic 1 side-by-side against solid tumor cell lines considering the significant impact we have shown here that the C(8)-OH stereocenter has on the effects of cytotoxicity for 1. We believe that additional SAR campaigns seeded by the diene-ol core structure of 1 could provide further useful insights leading to a next generation ADC therapeutic lead.

Having, thus, described the invention, what is claimed is:

### REFERENCES

(1) Crews, P.; Kakou, Y.; Quinoa, E. J. Am. Chem. Soc. 1988, 110, 4365.
(2) McAlpine, J. B.; Chen, S. N.; Kutateladze, A.; MacMillan, J. B.; Appendino, G.; Barison, A.; Beniddir, M. A.; Biavatti, M. W.; Bluml, S.; Boufridi, A.; Butler, M. S.; Capon, R. J.; Choi, Y. H.; Coppage, D.; Crews, P.; Crimmins, M. T.; Csete, M.; Dewapriya, P.; Egan, J. M.; Garson, M. J.; Genta-Jouve, G.; Gerwick, W. H.; Gross, H.; Harper, M. K.; Hermanto, P.; Hook, J. M.; Hunter, L.; Jeannerat, D.; Ji, N. Y.; Johnson, T. A.; Kingston, D. G. I.; Koshino, H.; Lee, H. W.; Lewin, G.; Li J.; Linington, R. G.; Liu, M. M.; McPhail, K. L.; Molinski, T. F.; Moore, B. S.; Nam, J. W.; Neupane, R. P.; Niemitz, M.; Nuzillard, J. M.; Oberlies, N. H.; Ocampos, F. M. M.; Pan, G. H.; Quinn, R. J.; Reddy, D. S.; Renault, J. H.; Rivera-Chavez, J.; Robien, W.; Saunders, C. M.; Schmidt, T. J.; Seger, C.; Shen, B.; Steinbeck, C.; Stuppner, H.; Sturm, S.; Taglialatela-Scafati, O.; Tantillo, D. J.; Verpoorte, R; Wang, B. G.; Williams, C. M.; Williams, P. G.; Wist, J.; Yue J. M.; Zhang, C.; Xu, Z. R.; Simmler, C.; Lankin, D. C.; Bisson, J.; Pauli, G. F. Nat. Prod. Rep. 2019, 36, 248.
(3) Wang, L. and Hale, K. J. Org. Lett. 2015, 17, 4200.
(4) Morgan, J. B.; Mandi, F.; Liu, Y.; Coothankandaswamy; Jekabsons, M. K.; Johnson, T. A.; Sashidhara, K. V.; Crews, P.; Nagle, D. G.; Zhou, Y. Bioorg. Med. Chem. 2010, 18, 5988.
(5) Meyer, K. J.; Singh, A. J.; Cemeron, A.; Tan, A. S.; Leahy, D. C.; O'Sullivan, D.; Joshi, P.; La Flamme, A. C.; Northcote, P. T.; Berridge, M. V.; Miller, J. H. Mar. Drugs 2012, 10 900.
(6) Sonnenschein, R. N.; Johnson, T. A.; Tenney, K.; Valeriote, F. A.; Crews, P. J. Nat. Prod. 2006, 69, 145.
(7) Sashidhara, K. V.; White, K. N.; Crews, P. J. Nat. Prod. 2009, 72, 588.
(8) Johnson, T. A.; Sohn, J.; Inman, W. D.; Estee, S. A.; Loveridge, S. T.; Vervoort, H. C.; Tenney, K.; Liu, J. K.; Ang, K. K. H.; Ratnam, J.; Bray, W. M.; Gassner, N. C.; Shen, Y. Y.; Lokey, R. S.; McKerrow, J. H.; Boundy-Mills, K.; Nukanto, A.; Kanti, A.; Julistiono, H.; Kardono, L. B. S.; Bjeldanes, L. F.; Crews, P. J. Nat. Prod. 2011, 74, 2545.
(9) Sugiyama, H.; Yokokawa, F.; Shioiri, T. Org. Lett. 2000, 2, 2149.
(10) Sugiyama, H.; Yokokawa, F.; Shioiri, T. Tetrahedron 2003, 59, 6579.
(11) Le Flohic, A.; Meyer, C.; Cossy, J. Org. Lett. 2005, 7, 339.
(12) Le Flohic, A.; Meyer, C.; Cossy, J. Tetrahedron 2006, 62, 9017.
(13) Amans, D.; Le Flohic, A.; Bellosta, V.; Meyer, C.; Cossy, J. Pure Appl. Chem. 2007, 79, 677.
(14) Serra, G.; Mahler, G.; Manta, E.; Heterocycles, 1998, 48, 2035.
(15) Rodriguez-Conesa, S.; Candal, P.; Jimenez, C.; Rodriguez, J. Tetrahedron Lett. 2001, 42, 6699.
(16) Mahler, G.; Serra, G.; Manta, E. Synth. Commun. 2005, 35, 1481.
(17) Rega, M.; Candal, P.; Jimenez, C.; Rodriguez, J. Eur. J. Org. Chem. 2007, 934.
(18) (a) Mahler, G.; Serra, G.; Dematteis, S.; Saldana, J.; Dominguez, L.; Manta, E. Bioorg. Med. Chem. Lett. 2006, 16, 1309-1311.
(19) Batt, F.; Fache, F. Eur. J. Org. Chem. 2011, 6039.
(20) Valeriote F. A.; Tenney, K.; Media, J.; Pietraszkiewicz, H.; Edelstein, M.; Johnson, T. A.; Amagata, T.; Crews. P. 2012, J. Exp. Ther. Oncol. 10, 119.
(21) Dess, D. B.; Martin, J. C. 1983. J. Org. Chem. 48, 4155.
(22) Dess, D. B.; Martin, J. C. 1991. J. Am. Chem. Soc. 113, 7.
(23) Trimurtulu, G.; Ohtani, I.; Patterson, G. M. L.; Moore, R. E.; Corbett, T. H.; Valeriote, F. A.; Lisa Demchik, L. 1994, J. Am. Chem. Soc. 116, 4729.
(24) He, H.; Ding, W.; Bernan, V. S.; Richardson, A. D.; Ireland, C. M.; Greenstein, M. Ellestad, G. A.; Carter, G. T. 2001, J. Am. Chem. Soc. 123, 5362.
(25) Bielitza, M. Pietruszka, J. 2013, Angew. Chem. Int. Ed., 52, 10960.
(26) Chen, H.; Lin, Z.; Arnst, K. E.; Miller, D. D.; Li, W. 2017, Molecules, 22, 1281.
(27) Jeffrey, S. C.; De Brabander, J.; Miyamoto, J.; Senter, P. D. ACS Med. Chem. Lett. 2010, 1, 277.

1. A compound of formula (I), formula (II), or pharmaceutically acceptable salt thereof, 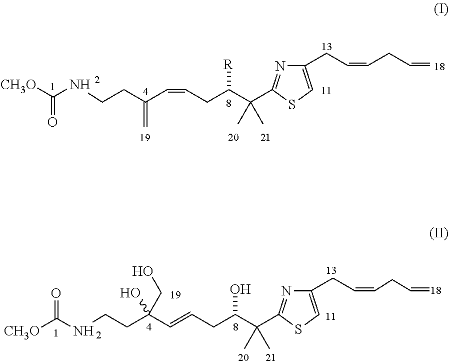
wherein R is OH, O-acetate, O—CH₃, F, Cl, Br, I, or an antibody.

2. A pharmaceutical composition comprising the compound or pharmaceutically acceptable salt thereof according to claim 1.

3. A method of treating cancer which method comprises administration of the pharmaceutical composition according to claim 2.

4. The method according to claim 3, wherein said cancer comprises pancreatic cancer, liver cancer, colon cancer, breast cancer, ovarian cancer, and lung cancer.

5. An isolation and storage method for extended shelf live (3-6 weeks) of the compound according to claim 1 for in vitro, in vivo and or clinical evaluation to prevent degradation.

6. The isolation and storage method of the compound according to claim 5 comprises:
a) purification and or transfer using aprotic solvents (>95% acetonitrile (by HPLC), dichloromethane, C₆D₆).
b) collection of purified solution of the compound in amber storage vials to prevent exposure to light.
c) evaporation of solvents surrounding the compound using Nitrogen stream to prevent oxidation.
d) storage of the compound under Argon in sealed vial, stored in dark desiccator with Dry-rite under vacuum.
